(19) United States
(12) Patent Application Publication
(10) Pub. No.: US 2017/0198246 A1
(43) Pub. Date: Jul. 13, 2017

(54) MULTIPURPOSE BIOREACTOR

(71) Applicant: Sarfaraz K. Niazi, Deerfield, IL (US)

(72) Inventor: Sarfaraz K. Niazi, Deerfield, IL (US)

(21) Appl. No.: 15/404,899

(22) Filed: Jan. 12, 2017

Related U.S. Application Data
(60) Provisional application No. 62/278,210, filed on Jan. 13, 2016.
(60) Provisional application No. 62/277,840, filed on Jan. 12, 2016.
(60) Provisional application No. 62/277,851, filed on Jan. 12, 2016.
(60) Provisional application No. 62/277,833, filed on Jan. 12, 2016.

(51) Int. Cl. C12M 1/00; C12M 1/36; C12M 1/34; C12M 1/12

(52) U.S. Cl. CPC C12M 23/28; C12M 37/02; C12M 29/20; C12M 41/48; C12M 47/18; C12M 23/26; C12M 29/26; C12M 29/18; C12M 47/10; C12M 41/12; C12M 47/12

(57) ABSTRACT

A multiuse bioreactor that is a single-use bioreactor, a development bioreactor, a commercial manufacturing bioreactor, a batch, a fed-batch, a perfusion and continuous bioreactor, a convective heat bioreactor, a product capture bioreactor, an ISO 9 bioreactor, a eukaryotic bioreactor, a prokaryotic bioreactor, a technology transfer-free bioreactor, and an inexpensive bioreactor is disclosed.

### CROSS REFERENCE TO RELATED APPLICATION

This application claims priority to U.S. Provisional Patent Application Ser. No. 62/278,210 entitled “OPTIMIZED CONTINUOUS RECOMBINANT PROTEIN EXPRESSION” filed on Jan. 13, 2016; U.S. Provisional Patent Application Ser. No. 62/277,840 entitled “FILTER-FREE BIOREACTOR EXHAUST” filed on Jan. 12, 2016; U.S. Provisional Patent Application Ser. No. 62/277,851 entitled MULTIPURPOSE UNIVERSAL BIOREACTOR filed on Jan. 12, 2016, and, U. S. Provisional Patent Application Ser. No. 62/277,833 entitled BIOREACTOR EXHAUST DECONTAMINATION, filed on Jan. 12, 2016, the contents of which are incorporated herein by reference in their entirety.

### BACKGROUND OF THE INVENTION

A bioreactor is a manufacturing device or system that supports a biologically active environment. In one case, a bioreactor is a vessel in which a chemical process is carried out involving organisms or biochemically active substances derived from such organisms. The bioprocess can either be aerobic or anaerobic. The use of bioreactors and their base design to manufacturing products for humans dates back to thousands of years. The modern bioreactor These use biotechnologies that began with the disclosure of the U.S. Pat. No. 2,535 issued on 1 Apr. 1842 to C. C. Edday titled Fermenting Vat. More recently, the design of bioreactor dates. The basic design of bioreactors has remained unchanged since the US patent for thousands of years, even though the control systems have continuously evolved. Commercial biomanufacturing dates back thousands of years from the first biological engine, the yeast, and continuing to the 2016 Nobel Prize winning application awarded to Yoshinori Ohsumi. Bioreactors are commonly cylindrical, ranging in size from a few liters to hundreds of thousands of liters, and are mostly made of stainless steel. A wide variety of cell-based prokaryotic and eukaryotic systems, as well as cell-free systems, have become available to us since then and we have expanded the line of products from yogurt, bread and wine to recombinant proteins, organs, vaccines and much more to come including products of individualized therapy. Based on the mode of operation, a bioreactor may be classified as a batch, fed-batch or continuous (e.g. a continuous stirred-tank reactor model). An example of a continuous bioreactor is the chemostat or perfusion bioreactor.

Like other technologies, biomanufacturing too has undergone evolutionary changes, but to meet the current challenge to control cost, a reinvention of the technology is required. The high cost of biological drugs is a result, in part, due to the high cost of development, ranging from up to $200 Million for a biosimilar product to more than $2.6 Billion for a new molecule. In 2015, the top ten $5B plus sales products included seven biologics, making this class of drugs highly lucrative resulting in many products being developed and companies investing billions into new facilities. However, these facilities were mostly the traditional types involving deep tank stainless steel reactors.

Organisms growing in bioreactors may be submerged in a liquid medium or may be attached to the surface of a solid medium. Submerged cultures may be suspended or immobilized. Suspension Bioreactors can use a wider variety of organisms, since special attachment surfaces are not needed, and can operate at much larger scale than immobilized cultures. However, in a continuously operated process, the organisms will be removed from the reactor with the effluent. Immobilization is a general term describing a wide variety of cell or particle attachment or entrapment. It can be applied to basically all types of biocatalysts including enzymes, cellular organelles, animal and plant cells. Immobilization is useful for continuously operating processes, since the organisms will not be removed with the reactor effluent, but is limited in scale because the microbes are only present on the surfaces of the vessel. Large scale immobilized cell bioreactors include moving media, also known as moving bed biofilm reactor (MBBR), packed bed, fibrous bed, and membrane types.

One of the more recent applications of bioreactors comes in the manufacturing of therapeutic products that constitute the most expensive life-saving and life-altering drugs. Reducing cost of manufacture of therapeutic products is a primary target of bio innovations to make these drugs more affordable. Regulatory agencies now allow commercialization of biological drugs is coming off patent as biosimilars with the aim to provide more affordable choices to patients. The new pathway involves demonstrating biosimilarity, a tiered approach to prove that the biosimilar drugs are highly similar to the innovator biological molecules. Biosimilarity demonstration is a complex exercise of matching the structural and functional similarity between the two molecules requiring extensive and expensive exercises in the development of biosimilar products, more particularly larger molecules such as monoclonal antibodies that are subject to subtle manufacturing variations such as glycosylation alterations, posttranslational modifications and the molecular variations (microheterogeneity) that can affect their potency and toxicity. Thus, the development cycle of biosimilars products is highly complex, time-consuming, and as a result very expensive.

There is an unmet need to design a bioreactor that will allow faster development of new biological products, less cumbersome scale-up to commercial scale, minimal studies required for transfer of technology and overall require low capital and running cost of manufacturing biological drugs intended for human use. The bioreactor should also be able to operate a variety of processes including batch, fed-batch, continuous, and perfusion.

Prior art in development scale bioreactors includes small volume micro bioreactors that have volume capacity in a few mL range. While some valuable information about the properties of the cell culture and its product can be obtained using micro bioreactors, the exercise to determine optimal bioprocess conditions and scaling up the bioprocess remains a major hurdle in the ability of the developer to taking these products to market at a reasonable cost. The exercise of process condition optimization and scale-up requires hundreds of experiments that consume years and hundreds of millions of dollars. A cost-effective solution to the development of biosimilar products comes from the active conduct of complex design of experiment details as well as reducing the cost of infrastructure that requires using a single-use bioreactor system.

Moreover, this where a great dilemma arises; at an early stage, the developers desire to produce just sufficient quantity of a biological drug to test it in animals and perhaps in humans. However, even producing those small amounts requires conducting the exercises mentioned above, an also, operating bioreactors in GMP-compliant clean rooms, a step that is not only expensive but not available to small development companies and research institutions. It would be desirable if the bioreactor can be operated in ISO 9 environment while fully complying with all GMP requirements. A contamination-proof bioreactor is an unmet need. Finally, a commercial-scale bioreactor is required to produce large quantities of biological drugs; while, at this stage, a pharmaceutical company may be able to afford expensive facilities to accommodate these bioreactors, there remains an unmet need to provide a bioreactor system that can be operated in ISO 9 environment at a modular scale, wherein any quantity of biological drug can be produced without making investment in extensive facilities.

There remains an unmet need for a bioreactor that allows simulation of hundreds of bioprocess conditions simultaneously, allows large-scale commercial manufacturing, as well as operable under ISO 9 environment to reduce the cost and time to market for new and biosimilar biological drugs. The instant invention solves this problem by disclosing a single-use bioreactor system capable of conducting hundreds of design of experiment (DOE) studies simultaneously and scaling up at the same time, all in ISO 9 environment.

The key features of the instant invention comprise a shaking platform with a plurality of containers of variable sizes, to resolve hundreds of permutations and combinations of bioprocess conditions, including temperature, pH, nutritive additives, nutritive gas tension, and intensity of shaking. The claimed bioreactor further comprises a product capture apparatus to reduce the downstream steps, further expediting the time to market for biological drugs. Additionally, the bioreactor can be operated under ISO 9 environment.

A smart bioreactor, the subject of instant invention, is a single-use bioreactor, a development bioreactor, a commercial manufacturing bioreactor, a batch, a fed-batch, a perfusion and continuous bioreactor, a convective heat bioreactor, a product capture bioreactor, an ISO 9 bioreactor, a eukaryotic bioreactor, a prokaryotic bioreactor, a technology transfer-free bioreactor, and an inexpensive bioreactor is disclosed.

### BRIEF SUMMARY OF THE INVENTION

The present invention provides a bioreactor comprising a container capable of holding a liquid of varying volumes, having a top surface, a bottom surface, at least one liquid inlet in the top surface, wherein the liquid inlet includes a control valve and a connection to a source of culture medium, a source of pH altering solution and a source of feed additives, wherein each source has a liquid flow controller, which also includes a control valve; at least one gas inlet in the top surface comprised of a sterilizing filter, a gas flow controller for each source of gas which also includes a control valve, an inline heating, and cooling element, and a control valve, wherein the gas inlet is connected to a source of a nutritive gas and a source of an inert gas; at least one gas outlet in the top surface comprising a one-way valve and a variable-speed vent fan; at least one liquid outlet in the bottom surface consisting of a control valve; a plurality of sensors disposed inside the bioreactor capable of monitoring conditions present in the bioreactor such as pH, temperature, pressure, and concentration of dissolved gases in the culture medium; and at least one gas sparger connected to the gas inlet, wherein the sparger extends below the surface of the liquid.

The container is additionally coupled to a capture column connected to the liquid outlet of the container, wherein the capture column comprises a product binding medium, a control valve located between the liquid outlet and the capture column, and a solution inlet having a control valve to enter solutions to wash and elute bound product in the capture column. The resin in the capture column may be divided into a plurality of porous pouches of such porosity that the resin does not escape the pouches and wherein the pouches are separated by a series of porous plastic plates comprising a gasket between each of the porous plastic plates. The perforated pouches may be made of nylon membrane consisting of pores ranging in size between 5 and 50 microns.

A plurality of sensors display conditions of culture medium on a display screen, and an electronic device controls the valves, liquid and gas flow apparatus to maintain a pre-determined physicochemical condition in the container.

A support platform to hold the container comprises side walls a circular its periphery and moveable partitioning walls resulting in multiple compartments, wherein each compartment can support a container, and wherein the containers may be secured to the support platform or the partitioning walls; the support platform has a hole corresponding to each container disposed on it to allow the liquid outlet to pass through and connect to the capture column that hangs underneath the support platform. The culture medium flows out of the container and into the capture column under gravity flow.

The bioreactor of the present invention allows instant capture of expressed product as the culture medium can flow through a capture column, removing at least two downstream steps: cell separation by centrifugation and filtration to reduce volume; both steps affect the quality and quantity of expressed product, creating a high level of variability in the molecular structure that makes DOE studies difficult to conclude. At a commercial degree of manufacturing, an early capture step provides consistency, higher yield, reduced safety risk in the batches products.

The containers in the instant invention are disposed on a single support platform; wherein they are compartmentalized to prevent them from striking each other. Each container is filled with a pre-determined volume of culture medium, filling. Half to two-third of the internal volume of the container and the medium is warmed to the proper growth temperature by bubbling a nutritive gas that has been preheated, a feature unique to the instant invention. In prior art, multiple reaction vessels are not allowed to be operated simultaneously, and no measure is available for adjusting the temperature of culture medium for each vessel; further, by heating the culture medium using gas flow, instead of a heated contact surface, temperature variability in the activity of the cell culture is minimized resulting in substantially higher productivity of the cell culture as well as consistent expression of products. With each container now equipped with its heating mechanism that allows for temperature adjustment, a cycling of temperature, low to high to low, can now be studied simultaneously to identify an optimal bioprocess cycle in one experiment. Additionally, bioprocessing conditions such as pH, medium composition and a nutritive load of the culture medium can be altered in each container independently, allowing the study of many permutations and combinations of bioprocess conditions simultaneously. Once the product has been produced, the contents of the container flow through a capture column containing a product binding resin, as a culture medium and cell debris flows through the column. The bound product can be washed in the capture column and eluted for further studies. The instant invention providing quick capture of expressed product without subjecting the product to any conditions of downstream process removes a major source of variability in product structure to allow a better and quick understanding of the effect of various bioprocess conditions. The prior art is silent on a bioreactor that will allow fast development of a bioprocess cycle as disclosed in the instant invention.

The instant invention allows simultaneous deployment of containers of different sizes on the same platform, giving additional information about scale-up complexities, while each container is subjected to the same conditions of shaking. Since containers of all sizes are subjected to same shaking conditions, a better understanding of the impact of scaling-up on the quality of expressed product is gained.

While the instant invention allows a faster throughput of development, the claimed bioreactor can be operated in an ISO 9 environment that is much cheaper to construct and operate; this is made possible by sealing all ports of the containers with sterile filters, preventing any contamination from entering the container or the contents of the exhaust of the container contaminating the environment. While most commercial manufacturers will prefer to operate these bioreactors in the more controlled environment to satisfy the regulatory agency requirements, these conditions will be fully acceptable to regulatory agencies, at least for the manufacturing of clinical supplies, making it possible for many smaller institutions and companies to generate high quality of product drugs for testing purpose.

The claimed bioreactor is a development bioreactor, a commercial bioreactor and a bioreactor capable of operating under ISO 9 conditions, all adding to cost-reduction, fast throughput of development and commercialization of product drugs.

### BRIEF DESCRIPTION OF THE SEVERAL VIEWS OF THE DRAWINGS

FIG. 1 is a cross section of the elements and features of the product expression bioreactor and capture column.

FIG. 2 is a cross section of the elements and features of the capture column.

FIG. 3A is a perspective view of the solid surface with containers.

FIG. 3B is a view of containers 1 disposed side-by-side on the 34: support surface

FIG. 3C is a single container 1 disposed on the 34: support surface

FIG. 4 is a topical view of the solid support,surface showing a plurality of compartments.

### DETAILED DESCRIPTION OF THE INVENTION

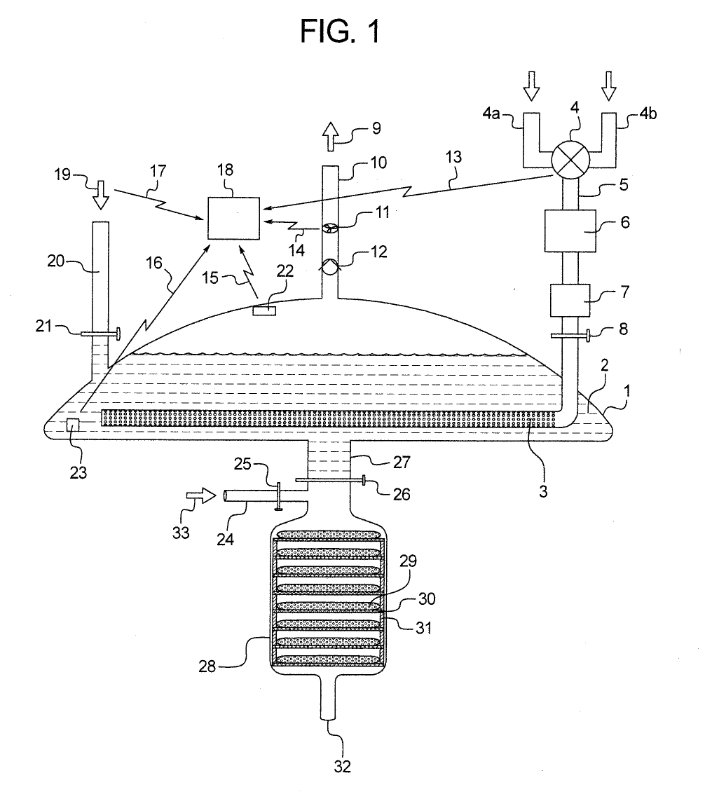

The core component of the invention involves a container capable holding a culture medium and cell culture to express biological products that are instantly harvested by allowing the culture medium to flow through a capture column. FIG. 1 shows a cross-section of the design and the key elements of the claimed bioreactor; 1: Container; 2: Culture medium and cell culture; 3: Tubular gas sparging unit; 4: Gas mixing valve; 4a: Nutrient gas inlet; 4b: Inert gas inlet; 5: Gas inlet; 6: Inline gas heater or cooler; 7: Inline gas sterilizing filter; 8: Gas inlet control valve; 9: gas exhausted to environment outside the room of operation, optionally through a Bunsen burner (not shown); 10: Exhaust gas outlet; 11: Inline vent fan; 12: One-way exhaust gas flow control valve; 13: Connection of gas mixing valve to electronic controller; 14: Connection of inline vent fan to electronic controller; 15: Connection of pressure sensor 22 to electronic controller; 16: Connection of culture media sensors 23 to electronic controller; 17: Connection of source of liquid 19 to electronic controller; 18: Electronic controller; 19: Source of liquids; 20: Liquid inlet; 21: Liquid flow control valve; 22: Pressure sensor; 23: Plurality of sensors for temperature, pH, nutrient gas tension and cell titer count; 24: Process liquid inlet; 25: Process liquid control valve; 26: Terminal outlet control valve; 27: Terminal liquid outlet; 28: Capture column; 29: Flexible porous pouch holding a binding resin; 30: Perforated hard surface; 31: Gasket; 32: Process liquid outlet.

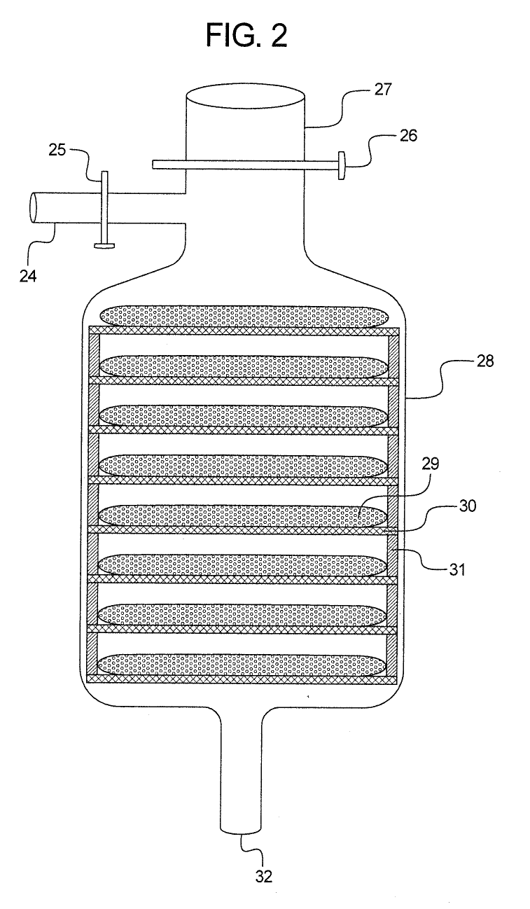

FIG. 2 shows a cross-section of the details of the capture column. 24: Process liquid inlet; 25: Process liquid control valve; 26: Terminal outlet control valve; 27: Terminal liquid outlet; 28: Capture column; 29: Flexible porous pouch holding a binding resin; 30: Perforated hard surface; 31: Gasket; 32: Process liquid outlet.

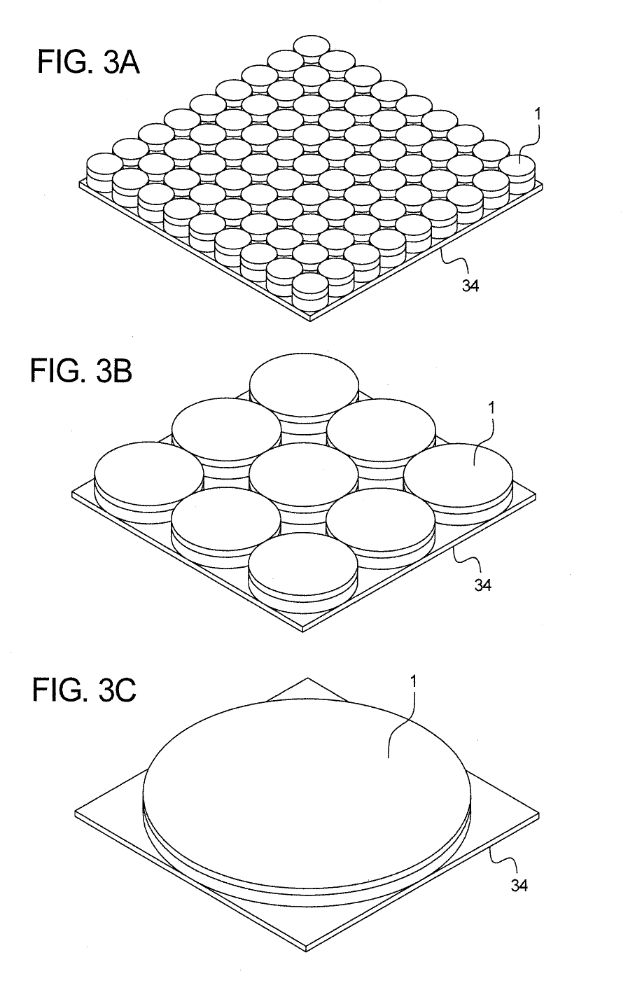

FIG. 3 show a perspective view of the support surface disposed with a singular and a plurality of containers. FIG. 3a: 1: containers disposed side-by-side on the 34: a support surface; FIG. 3b: 1: containers 1 disposed side-by-side on the 34: support surface, 3c: 1: a single container 1 disposed on the 34: support surface.

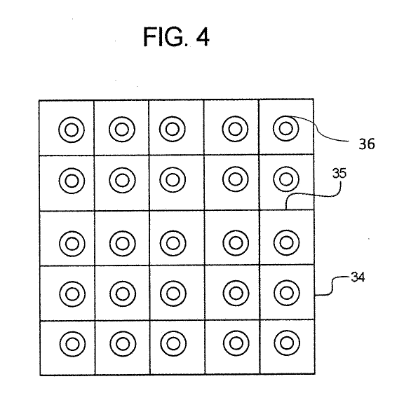

FIG. 4 shows a topical view of the 34: support surface with 35: partitions and side walls 35 and 36: holes to allow the Terminal liquid outlet to pass through the support surface 34.

The container of the instant invention is most suitably a flexible pillow-type container that is disposed on a support platform, capable of orbital rotating, linear shaking, vibrating, or a combination thereof. This motion is necessary to provide mixing of the content of the container. Unlike traditional bioreactors, both single-use, and deep-tank fixed wall bioreactors, a plurality of experiments can be conducted simultaneously for development as well as commercial manufacturing. When used as a development bioreactor, the plurality of containers hold culture medium with different physicochemical properties, and when used as a commercial bioreactor, the plurality of containers hold culture medium with similar physicochemical properties. In the latter case, the captured product from each container is mixed to create a single batch. The instant invention is a development and commercial manufacturing bioreactor that is suitable for almost all applications of a bioreactor intended to manufacture biotechnology products including biological products.

In one embodiment, the instant invention is a Single-Use Bioreactor to overcome the risk of cross contamination in using deep tank technology resulting from the installed utilities, steaming-in-place, cleaning-in-place procedures as well as complex cleaning validation exercises between batches. These requirements in traditional manufacturing using deep tank technology add substantial cost and time in the development and manufacture of biological drugs. The instant invention reduces the high cost of biological drugs by reducing the risk factors in development and manufacturing by utilizing only single-use containers that can have hard walls or soft-walls, constructed out of any material that can be sterilized prior to use including plastic, metal, or a composite. Additionally, the container may be square, rectangular or round. The round shape is the preferred shape wherein the diameter may range from 5 to 400 inches. The preferred motion for the round container is an orbital motion to reduce inconsistencies resulting from variations in shapes and sizes. With only one side as found in a round container, the turbulence due to corners found in the containers is obviated, allowing smooth movement of culture medium throughout the bioprocess cycle. The containers may have different capacity and are filled to 30-70% of capacity with the culture medium; the volume of culture medium can range from 10 mL to 1000 L, and a plurality of containers may be disposed simultaneously on a support surface. The shape and size of the container can significantly alter the nature of product expressed, particularly, when using a eukaryotic organism, where post-translational changes are anticipated. For example, a rectangular shaped container may provide a different yield and glycosylation pattern than a square or a round container, complicating the nature of scale-up studies involved. The instant invention resolves these difficulties by using a same shape of the container to conduct all studies, regardless of the size of the container. As containers are disposed side-by-side, an instant understanding of the effect of shape and size change in the container becomes evident early in the development stage.

In yet another embodiment, the instant invention is an ISO 9 Bioreactor. Bioreactors are operated under ISO 8 or lower environment conditions to minimize contamination. The instant invention creates an ISO 9 bioreactor by providing a pre-sterilized container that receives all components, liquid or gas, also pre-sterilized and removes the exhaust to the outside of the room minimizing the risk of contamination. By patenting all inlets and outlets of the container, it is now possible to operate the bioreactor in ordinary laboratory rooms without the need for clean rooms that require high capital investment to construct, and incur high maintenance cost of operating and validation. The ISO 9 bioreactor of the instant invention can be used in ISO 8 or the lower environment if required, making it possible thus allows development and at least manufacturing of initial clinical supplies in smaller laboratories not able to afford clean rooms to achieve this goal. Since the bioreactor is completely sealed, it is possible to operate multiple bioreactors within the same room and develop or manufacture different products simultaneously or at least allow the use of the same facility to manufacture various products concurrently. In some instances, the manufacturer may want to move the equipment to an ISO 7 environment where required by regulatory agencies.

In yet another embodiment, the instant invention is an Efficient Tech Transfer Bioreactor. Technology transfer of a bioprocess is a highly complex process. The regulatory agencies require the manufacturers to conduct a formal Comparability Protocol to demonstrate that the quality of the bioprocess is not altered when the technology is transferred, from one location to another. The instant invention eliminates the need for conducting this protocol since the complete bioreactor is transferrable to another location, without being dependent on any location-specific element, such as utilities, systems for clean-in-place or sterilize-in-place requirements. This feature of the claimed invention not only saves considerable cost, but it also promises to provide products of higher consistency.

In yet another embodiment, the instant invention is a Convective Heat Transfer Bioreactor. The current prior art of heating or cooling the content of a bioreactor involves conductive heat transfer whether it is an unlined stainless steel vessel or lined with plastic, such as in the case of single-use types. Jacketed vessels are most commonly used and in some instances, an electrically heated metallic platform is used to transfer heat. The conductive method of heat transfer is inherently inefficient, even when used in a metallic container because the GMP considerations require the use of stainless steel, which is inherently inefficient for heat transfer. The thermal conductivity [k, W/(m K 1 W/(m K)=1 W/(m° C.)=0.85984 kcal/(h m° C.)=0.5779 Btu/(ft h° F.)=0.048 Btu/(in h° F.)] of vacuum is zero, for carbon it is 1.7, for stainless steel it is 16, for iron it is 80, for aluminum it is 200, for silver it is 429 and for diamond it is 1000. The use of stainless steel is preferred to reduce leaching and extractable components, to offer ease of cleanliness, and to provide an esthetic appearance. However, the poor conductivity of stainless steel makes it a poor choice for bioreactors since the temperature gradient is small, as bioreactors are rarely heated above 40° C. This disadvantage becomes more noticeable if it is desired to cycle the temperature, such as within a few degrees—a quick adjustment of temperature in a stainless-steel vessel is not possible. Additional difficulties arise when the stainless-steel vessels are lined with plastic that constitutes the single-use element of the bioreactor, or where a plastic bag is used in a free-standing design. The conductivity of polyethylene, both low density and high density is less than 0.5, making it the worse choice for heat transfer.

There is a serious unmet need to overcome the inefficiencies in heat transfer in bioreactors and it is fully resolved in the instant invention by creating a convective system, wherein a heated gas is used to provide heat transfer. First, the incoming nutritive gasses are heated or cooled, as necessary, to a temperature equal to or slightly higher or lower (in heating or cooling) before they contact with the culture medium. Second, where thermal energy imparted by the nutritive gasses does not provide sufficient heat transfer, an inert gas supply is provided to supplement the heat transfer; the inert gas can also be used alone for this purpose. The convective approach, in contrast with the conductive approach, provides a faster equilibration of the temperature of culture medium, eliminates thermal shock to the cell culture, allows faster temperature cycling and has the added ability to heat or cool the culture medium on demand. There is no prior art in the use of single-use bioreactors that allows convective temperature modulation of the culture medium. The gas supplied into culture medium may be maintained at an appropriate growth temperature for the chosen cell line, for example between 20-40° C. The gasses entering the bioreactor may also be heated or cooled to 2-5° C. higher or lower, when cooling, than the desired culture medium temperature to allow easier adjustment of temperature. The culture medium can be heated or cooled with the gas before adding the cell culture to avoid thermal shock to the cells. Typically, the nutritive gas is oxygen or carbon dioxide. In some case, particularly when exploiting eukaryotic cells, the amount of nutritive gas will not be sufficient to maintain the desired temperature in the container requiring the use of inert gas to provide temperature adjustment. The inert gas may be nitrogen or a noble gas.

This feature of gasification in the instant invention makes it possible for each container to operate independently when a plurality of containers is operated simultaneously, such as when conducting the DOE exercises. No prior art in the design of development bioreactors allows, first, use of multiple containers operating on the same platform, so they are subjected to same physical movement, and second, allowing temperature adjustment independently for each container.

An additional attribute of the instant invention allows almost immediate readjustment of the temperature of culture medium, where a temperature cycling is required. It is often seen more productive, for example, to switch the temperature from 37° C. to 32° C. to 37° C. to obtain a desired post-translational modification. Such fine adjustment of the temperature of the culture medium is rarely possible when using a single-use plastic container because of its poor conductivity; this limitation is removed in the instant invention.

In yet another embodiment, the instant invention is a kLa Optimized Bioreactor. Gasification of culture medium to maintain the proper nutritive tension of gasses requires fast dispersion of nutritive gasses and efficient removal of metabolic gasses; one without other results in a poor environment for the growth of cells. In prior art, gasification is provided by a single point sparging such as in deep tank bioreactors, with or without single-use liners, and through surface aeration, in some single-use bioreactors. The former approach requires high shear force to distribute gas within the bioreactor, while the dwell time of metabolic gasses remains high because of the vertical disposition nature that requires longer travel distance; both attributes produce inconsistent pH control, as well as nutritive gas tension. In the latter approach, the device can only be used for eukaryotic cells, is limited to smaller sizes and surface renewal is often unpredictable. The unmet need for optimized KLA in a bioreactor is provided in the instant invention by providing a tubular sparging element disposed across the entire length of the container to provide a larger surface area for gasification and by disposing the container horizontally, a smaller pathway for the escape of metabolic gasses. The sparging element may constitute a plurality of tubular elements spread across the base of the container, particularly, when the size of the container is large. The sparging unit will ideally have perforations ranging from 1-100 microns. The size of perforations can be variable, from smaller to largest, as the distance from the point of introduction of gas, to assure that the pressure in the unit is maintained throughout its length and thus, the volume of gas going out of the unit across its linear surface. The material for tubular elements can be ceramic, plastic or metal.

In yet another embodiment, the instant invention is a Horizontal Bioreactor. Commercial bioreactors are vertically disposed; single-use bioreactors in commercial use are lined vertical bioreactors, similarly disposed; the Wave bioreactor, which is a horizontal bioreactor is only useful for mammalian cells as it lacks a sparging system and is not scalable to commercial volumes. The instant invention provides a solution for the unmet need for a bioreactor that allows installation in rooms with low ceilings, opening the utility to research institutions, small companies, and worldwide to the development of drugs and vaccines at an affordable cost while allowing their use for all types of cells and organisms. Additionally, by disposing the bioreactor in a horizontal direction, the instant invention allows conducting multiple studies simultaneously for DOE purpose. A horizontally displaced bioreactor also offers a very short path for the metabolic gasses to escape, providing better control of pH, improve kLa and much-reduced need to agitate culture medium to mix gasses.

In yet another embodiment, the instant invention is a Variable Size Bioreactor. The instant invention provides bioreactor platform that can be disposed with different volumes, from a few mL of thousands of liters of culture medium, while using the same geometry of the container, and the same physical movement to allow a faster scale-up and optimization of bioprocess conditions. Unlike the prior art, the instant invention allows operation of different size containers simultaneously on a single platform allowing quick comparison of the impact of scaling up. The prior art provides only fixed capacity bioreactors that require multiple size platforms to provide different capacities since using different volumes in the container changes the geometry of mixing and thus does not provide the efficiency analysis. In the instant invention, the containers are always filled to an optimal volume, from 50% to 70%, yet represent a variety of volumes. The instant invention provides a solution to these unmet needs by providing a single platform that can be used with different size of the containers, and the number of containers deployed simultaneously will only depend on the available floor area. Since the support platform is rotated orbitally, shaken, or vibrated and the prior art suggests available motorized devices move tons of material, the limitations on the size of support platform are not limiting. There can, however, be some restrictions on the size of the container, if a flexible plastic bag is used, in which case, a plurality of bags of maximum useful size are operated side-by-side and their yield combined at the end of the cycle as allowed in GMP manufacturing.

In yet another embodiment, the instant invention is a Bioprocess Development Bioreactor. An optimal bioprocessing protocol includes three steps: first, selecting a right cell culture strain and secondly, a right set of conditions that yield a product of desired post-translational characteristics and third, a consistent expression under the condition of variability that is inevitable in commercial manufacturing. One can readily see that with many variables, and their permutations and combinations, the number of studies required to complete these three steps can easily amount to hundreds—an expensive task.

To avoid these costs, most developers only use a limited number of attributes to optimize the bioprocess, risking not determining ideal and cost-effective conditions as the DOE exercise is made selective, assuming the certain risk of not being able to optimize accurately. The unmet need of providing a platform that will allow the conduct of DOE with many experiments simultaneously is resolved in the instant invention. The instant invention provides a single platform capable of housing a plurality of containers, which can be different sizes; each container can be operated under different conditions. A 10-ft×10-ft platform can work 400 containers of 6-inch diameter; this footprint of the platform is small enough to be accommodated in any size laboratory. ***The number and size of the bioreactor used for optimizing bioprocess conditions may be calculated by conducting a Design of Experiment (DOE) exercise using variable factors as the cell culture strains, the types, and volumes of culture medium, the pH, the temperature and cycling of temperature, the culture medium additives added initially or periodically to the culture medium. After conducting such experiments, the conclusion of results can lead to selecting a strain of cell culture most suitable for commercial production. As an example, Table 1 shows a Design of Experiment (DOE) approach to conducting an evaluation that will result in the selection of a strain of a cell culture, bioprocess conditions and scale-up risks simultaneously. Conducting over 400 experiments (Table 1) would require a long time and high expense using the prior art. By allowing a side-by-side testing, it is now possible to optimize several conditions including compositions of culture medium, pH, temperature, concentration of culture medium additives and nutritive and inert gasses supplied into the bioreactor, or other numerous parameters. Prior art using micro bioreactors are all based on a well or a plate design and do not allow scale-up to commercial production, are mostly limited to bacterial cultures and have limited value in the development of products with post-translational modifications.

In yet another embodiment, the invention includes a support platform comprising side walls around its periphery and moveable partitioning walls resulting in multiple compartments, wherein each compartment can support a container, and wherein the containers may be secured to the support platform or the partitioning walls. The supporting platform further provides holes in its surface to allow passage of the liquid outlet of the container and connection to the capture column, to allow a simple gravity flow of the culture medium without the need to subject the culture medium to the stress of a peristaltic pump that has the potential of damaging the expressed product. A mechanical device is connected to the solid surface comprises a motor and a set of gears capable of providing an orbital motion to the solid surface ranging from 1-50 rpm, vibrating or horizontally shaking the solid support surface.

TABLE 2
Calculation of number experiments for cell culture strain selection,
optimization of product expression and scale-up to a commercial level.

| | | Cumulative Experiments |
|---|---|---|
| Attribute | Variants | (# of containers) |
| Cell Line Strain | 3 | 3 |
| Medium Composition | 3 | 9 |
| Temperature | 2 | 18 |
| Medium additives | 6 | 108 |
| pH | 2 | 216 |
| Scale up | 2 | 432 |

In yet another embodiment, the instant invention is a Commercial Bioreactor. Historically, the commercial bioreactors are deep tank stainless steel types that have volume capacity into hundreds of thousands of liters; the financial advantages of such large capacity bioreactors has fallen into ill-repute for two reasons; first, if such large volume bioreactors are contaminated, the entire batch is discarded, costing millions of dollars and secondly, the sharp increase in the cell culture yields that are now 10 to 100 times what they used to be just a couple of decades ago have made these traditional bioreactors obsolete. However, the art of single-use bioreactors has not progressed enough to make them a clear alternative to the traditional deep tank bioreactors. The instant invention fills the gap in this unmet need by two distinct features. First, being a horizontal bioreactor, it is more cost-effective to supply a plurality of bioreactors than increasing the height of the bioreactor, and secondly, the ability of the instant invention to capture the expressed product from each separately into smaller volumes of solution allows mixing of the yields from a plurality of bioreactors at the end of the cycle to create a larger batch size. An additional advantage of this approach comes from the option of discarding those yields that are contaminated, reducing the cost of risk substantially. This method of combining the output of a plurality of bioreactors follows CFR 21 regulations that define a batch. No prior art allows for this flexibility of modularity, in line capture and creating an infinitely variable batch without having to validate different sizes of bioreactors.

In yet another embodiment, the instant invention is a Capture Bioreactor that works concurrently with product expression. The expressed product is captured in a column device attached to the liquid outlet of the container. The capture column can take any form suitable to bind the expressed products such as by holding a suitable amount of a resin specific to binding the expressed product. However, to make the capture step work consistently, a design feature is introduced in the capture column. The binding resin is contained in a plurality of porous pouches capable of holding the resin inside the pouch by having a porosity which is smaller than the particle size of the binding resin. A plurality of pouches is separated from each other first resting the pouches on porous plastic plates that are spaced by a gasket disposed between the porous plastic plates to prevent compression of the pouches. The perforated pouches may be made of a nylon membrane wherein the size of porosity ranges between 5 and 50 microns. The quantity of binding resin in the capture column is calculated based on the binding capacity and the amount of product to be removed from the culture medium. The expressed product is captured in the column by letting the content of the container flow through the capture column, wherein the product binds to a resin disposed in the column, washing the product resin complex with a cleaning solution to remove cells, debris and other chemical entities, followed by eluting the product from the product resin complex in the capture column by passing an elution solution through the capture column to collect a concentrated solution of the expressed products for immediate studies or further purification. Traditional methods of product capture involve a first step to remove the cells by a high-speed centrifugation process and then, a filtration step to reduce the volume of culture medium prior to subjecting it to purification, both steps causing damage to product due to stress reducing the yield, as well as, bruising and battering the product to alter its structure resulting in an unfortunate comparison of product structure arising from changes in the bioprocess conditions.

The current art involves the use of filters to isolate the product from the culture medium and cell culture that is subject to high risk of contamination, distress to the product in the process of filtration risks contamination of bioreactor, and requirement of expensive equipment. Additionally, the cost of hardware and operation of these two universal steps is very high; additional cost comes from the lengthening of bioprocess cycle, for about 40-50 hours, that all add to the cost of development. The instant invention cures all of these unmet needs for cost reduction by providing a capture column containing a resin capable of binding the expressed product; as culture medium passes through the capture column, only the product is retained, and the culture medium and cell culture are drained out. The instant invention also provides for meeting a similar unmet need when organisms are used wherein the product is expressed by the organism as an inclusion body, such as in the case of prokaryotic organisms, wherein, the inclusion bodies are first solubilized in the container, before passing them through the capture column. Being able to process both eukaryotic and prokaryotic organisms, in yet another embodiment, the instant invention is Universal Cell Bioreactor, in yet another embodiment, the instant invention is universally applicable to all types of cells and organisms. The bioreactor may be used to express any biological product in any host cell, including a bacterium, yeast, a mammalian cell, a plant cell, a tissue cell, a virus, or a fusion cell. The recombinant cell may carry multiple gene modifications making it capable of expressing a plurality of products. The current technology is highly subject to the type of cell or organism used to produce products. An unmet need is to provide a universal bioreactor capable of growing all kinds of cells and organisms. The instant invention cures this unmet need by providing a platform that can provide any level of gasification because of its filter-free exhaust system, the type and degree of agitation and other features that make it possible to conduct any operation a bioreactor in the instant invention.

In yet another embodiment, the instant invention is a Filter-free Exhaust Bioreactor. Sterilizing filters in the air exhaust are used to prevent contamination of the clean rooms where bioreactors are operated; when using flexible containers, this creates additional risk of contamination of the bioreactor due to a back pressure from blocking of filters has prevented their use on a commercial basis; removing filters and exhausting the gases to outside environment resolves the risk of contamination, over pressurization, yet assures no return of any air back into containers and room contamination.

The instant invention provides a solution to this unmet need by removing filters in the exhaust and instead replaces them with a mechanical system that exhausts gasses to outside environment while assuring that no air gets back into the bioreactor. The instant invention achieves this by first, providing a one-way valve and a shut-off valve as a precautionary measure. Also, a vent fan triggered by a pre-determined pressure in the container keeps exhausting the gas to the outside of the room. It is a combination of several features of the instant invention that allows the bioreactor to be operated in an ISO 9 environment, without any risk of contamination of the content of the container as well as the room surfaces where the bioreactor is operated.

To further assure that exhaust gas does not contaminate the environment within or outside of the room, the exhaust gas is passed through a Bunsen burner with a source of fuel gas and oxygen to burn the exhaust gas contents, including any living cells that might be carried with droplets. A source of oxygen is needed since the exhaust gas is mostly stripped of oxygen.

In yet another embodiment, the instant invention is a Scaling Bioreactor. One of the most frustrating problems in the manufacture of biological products is that once the size of the bioreactor is increased, there are no guarantees that the product expressed will be the same as expressed in a smaller size bioreactor. This anomaly comes from a lack of reproduction of thermodynamic conditions when the dimensions of the bioreactor are changed. The instant invention cures this unmet need by providing two solutions; first, by allowing the pooling the captured product from one size of bioreactor to create a larger batch and second, by allowing testing of a multitude of sizes of containers side-by-side to determine if there are any changes in the product structure as the scale of the bioreactor changes.

In yet another embodiment, the instant invention is a Fed-batch Bioreactor; wherein the container is equipped with feed mechanism to allow fed-batch operation, while it can also be operated at Batch Bioreactor.

In yet another embodiment, the instant invention is a Perfusion Bioreactor. Biological products are expressed using a batch process, fed-batch process or a perfusion process; in the latter process, the fresh culture medium is provided as the expressed product is removed from the bioreactor; this requires a filtration process that returns the cell culture back to the bioreactor while removing culture medium. In prior art, perfusion methods require removal of culture medium from the bioreactor, passing through a filter to retain cells and discard culture medium, which is replaced with fresh culture medium.

The instant invention focused on operating under ISO 9 condition requires a different approach as provided in the instant invention by keeping the filter within the closed container, wherein the filter device comes with the container pre-sterilized and stays free of contamination. The filter provided in the instant invention pertains mainly to mammalian cells that range in diameter from 14-15 microns. The filter comprised a ceramic element, similar to the sparging rod with a diameter of fewer than 10 microns and disposed next to the sparging unit, who flow of air keeps the filter unclogged. Additionally, the filter can be electrically charged by external means to repel the mammalian cells away from the filter to prevent blockage. A variety of arrangements of a plurality of ceramic rods can be arranged to optimize the filtration of culture medium to retain the cells. Since perfusion volumes are much smaller and the requirement of replacement of culture medium vis-à-vis total volume of culture medium is small, it is possible to allow draining of the culture medium under gravity, and alternately using a peristaltic pump installed between the liquid outlet and the capture column.

In yet another embodiment, the instant invention is a Continuous Bioreactor. The current art of perfusion bioreactors provides a smaller size bioreactor to produce higher quantities of the product; the volume of culture medium, which is a source of carbon, remains the same, expect, instead of a larger volume, a smaller size bioreactor can be used to produce proportionally higher quantities of product. It is a well-established fact the productivity of cell culture his highly dependent on their age when added to culture medium. The cell culture is most productive between 7-14 days of age in the culture medium, and it is for this reason that a batch process is terminated within this interval of time.

The current art is silent on how to keep a bioreactor in its most productive stage for a longer period, perhaps ad infinitum. The instant invention fulfills this unmet need by providing a bioreactor that can maintain a pre-determined average age of the cell culture in the bioreactors. This is provided by replacing both the culture medium as well as the cell culture continuously from the containers. The average age of the cell culture is determined by the fraction of the content of the containers removed continuously. Table 3 shows the calculation of the average age of the cell culture based on geometric dilution and exponential decay of the number of cell culture. The calculations are made using an exponential decay of the cell culture and presented as change daily. It is abundantly clear that a culture medium can be maintained the most desirable average age of the cell culture to express products continuously, ad infinitum. There is no prior art to provide an optimal expression of products ad infinitum. An additional advantage of the instant invention is that once a bioreactor has been setup, it can continuously produce the desired product on a continuous basis.

Calculation for relating average age of cell culture with the percent of culture medium (and therefore the percent of cell culture) removed per day from a container is given as follows:

Cell Culture Age (CCAge)=Σ(CCAgeₙ₋₁-F*CCAgeₙ₋₁)+1; the n=number of the day, at large n, the age is at large value of n, CCAge=F/100, or fraction to be removed per day to achieve a certain age upon equilibrium is 100/CCAge. Table3 shows the results of calculation, demonstrating the achievement of steady state (99%), which can be calculated by following equation: Days to steady state=7*(0.693/F).

TABLE 3
Cell culture replacement (per day) and the average age
of the cell culture (in days) in the bioreactor.

| | Age, No | Age, 5% | Age, 7.5% | Age, 10% | Age, 15% | Age, 20% |
|---|---|---|---|---|---|---|
| Day | Exchange | Exchange | Exchange | Exchange | Exchange | Exchange |
| 0 | 0.00 | 0.00 | 0.00 | 0.00 | 0.00 | 0.00 |
| 1 | 1.00 | 1.00 | 1.00 | 1.00 | 1.00 | 1.00 |
| 2 | 2.00 | 1.95 | 1.93 | 1.90 | 1.85 | 1.80 |
| 3 | 3.00 | 2.85 | 2.78 | 2.71 | 2.57 | 2.44 |
| 4 | 4.00 | 3.71 | 3.57 | 3.44 | 3.19 | 2.95 |
| 5 | 5.00 | 4.52 | 4.30 | 4.10 | 3.71 | 3.36 |
| 6 | 6.00 | 5.30 | 4.98 | 4.69 | 4.15 | 3.69 |
| 7 | 7.00 | 6.03 | 5.61 | 5.22 | 4.53 | 3.95 |
| 8 | 8.00 | 6.73 | 6.19 | 5.70 | 4.85 | 4.16 |
| 9 | 9.00 | 7.40 | 6.72 | 6.13 | 5.12 | 4.33 |
| 10 | 10.00 | 8.03 | 7.22 | 6.51 | 5.35 | 4.46 |
| 11 | 11.00 | 8.62 | 7.68 | 6.86 | 5.55 | 4.57 |
| 12 | 12.00 | 9.19 | 8.10 | 7.18 | 5.72 | 4.66 |
| 13 | 13.00 | 9.73 | 8.49 | 7.46 | 5.86 | 4.73 |
| 14 | 14.00 | 10.25 | 8.86 | 7.71 | 5.98 | 4.78 |
| 15 | 15.00 | 10.73 | 9.19 | 7.94 | 6.08 | 4.82 |
| 16 | 16.00 | 11.20 | 9.50 | 8.15 | 6.17 | 4.86 |
| 17 | 17.00 | 11.64 | 9.79 | 8.33 | 6.25 | 4.89 |
| 18 | 18.00 | 12.06 | 10.06 | 8.50 | 6.31 | 4.91 |
| 19 | 19.00 | 12.45 | 10.30 | 8.65 | 6.36 | 4.93 |
| 20 | 20.00 | 12.83 | 10.53 | 8.78 | 6.41 | 4.94 |
| 21 | 21.00 | 13.19 | 10.74 | 8.91 | 6.45 | 4.95 |
| 22 | 22.00 | 13.53 | 10.93 | 9.02 | 6.48 | 4.96 |
| 23 | 23.00 | 13.85 | 11.11 | 9.11 | 6.51 | 4.97 |
| 24 | 24.00 | 14.16 | 11.28 | 9.20 | 6.53 | 4.98 |
| 25 | 25.00 | 14.45 | 11.43 | 9.28 | 6.55 | 4.98 |
| 26 | 26.00 | 14.73 | 11.58 | 9.35 | 6.57 | 4.98 |
| 27 | 27.00 | 14.99 | 11.71 | 9.42 | 6.58 | 4.99 |
| 28 | 28.00 | 15.24 | 11.83 | 9.48 | 6.60 | 4.99 |
| 29 | 29.00 | 15.48 | 11.94 | 9.53 | 6.61 | 4.99 |
| 30 | 30.00 | 15.71 | 12.05 | 9.58 | 6.62 | 4.99 |
| 31 | 31.00 | 15.92 | 12.14 | 9.62 | 6.62 | 5.00 |
| 32 | 32.00 | 16.13 | 12.23 | 9.66 | 6.63 | 5.00 |
| 33 | 33.00 | 16.32 | 12.32 | 9.69 | 6.64 | 5.00 |
| 34 | 34.00 | 16.50 | 12.39 | 9.72 | 6.64 | 5.00 |
| 35 | 35.00 | 16.68 | 12.46 | 9.75 | 6.64 | 5.00 |
| 36 | 36.00 | 16.84 | 12.53 | 9.77 | 6.65 | 5.00 |
| 37 | 37.00 | 17.00 | 12.59 | 9.80 | 6.65 | 5.00 |
| 38 | 38.00 | 17.15 | 12.64 | 9.82 | 6.65 | 5.00 |
| 39 | 39.00 | 17.29 | 12.70 | 9.84 | 6.65 | 5.00 |
| 40 | 40.00 | 17.43 | 12.74 | 9.85 | 6.66 | 5.00 |
| steady- | n/a | 20.00 | 13.33 | 10.00 | 6.66 | 5.00 |
| state | | | | | | |
| Days to | n/a | 97 | 65 | 48 | 32 | 24 |
| 99% | | | | | | |
| steady- | | | | | | |
| state | | | | | | |

In yet another embodiment, the instant invention is a Cost-optimized Bioreactor as compared to prior art. Manufacturing of biological products requires an investment of hundreds of millions of dollars that keeps the smaller companies out of the competition, particularly, the manufacturing of biosimilars. The instant invention provides a cost-effective solution to the manufacturing or recombinant drugs involving a single-use platform that are infinitely scalable, functions in multiple formats and allows fast development and manufacture of commercial quantities of biological products.

The preferred embodiments described above do not describe every possible advantage of the claimed invention, as the user will find additional applications to specific needs of developing a new product. However, the instant invention brings together a multitude of features never operated before or having any obviously known utility to anyone. The key features include a single-use bioreactor, an easily transportable bioreactor, a bioreactor operable in ISO 9 environment, a bioreactor allowing hundreds of experiments to be conducted simultaneously, a bioreactor that provides test samples almost as soon as the bioreactor cycle is completed, a bioreactor that can be operated in low-ceiling height rooms, a bioreactor that is highly cost-efficient, a bioreactor that can be used to develop products expressed by all types of organisms, prokaryotic as well as eukaryotic, a bioreactor that allows alteration of bioprocess conditions such as temperature that had never been possible, a bioreactor that promises to allow development and manufacturing of biological drugs at large cost saving to manufacturers so they may pass on the cost benefit to patients.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein is merely intended to serve as a shorthand method of referring individually to each separate value falling within the range unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate the invention better and does not pose a limitation on the scope of the invention unless otherwise claimed. No language in the specification should be construed as indicating any non-claimed element as essential to the practice of the invention.

What is claimed is:

1. A bioreactor comprising:
a. at least one single-use container with an inner volume, capable of holding a cell culture and culture medium to express a biological product, a top surface, and a bottom surface;
b. at least one liquid inlet disposed in the top surface of the container in fluid communication with a plurality of sources of liquids including a culture medium, a cell culture, a pH adjusting solution, and a nutritive solution;
c. at least one gas inlet disposed in the top surface of the container and in fluid communication with a source of at least one nutritive gas and at least one inert gas;
d. an inline gas heater or cooler connected to the gas inlet;
e. an inline gas sterilizing filter connected to the gas inlet;
f. at least one gas sparging unit connected to the gas inlet and disposed in the culture medium;
g. at least one exhaust gas outlet disposed in the top surface of the container and in fluid communication with outside environment further comprising a one-way exhaust gas flow control valve and an inline vent fan;
h. a pressure sensor disposed in the container and connected to an electronic controller to adjust exhaust gas flow to maintain a positive pressure inside the container continuously;
i. at least one liquid outlet disposed in the bottom surface of the container, further comprising a liquid flow control valve;
j. a movable raised support platform to hold the container with an opening to allow passage of the liquid outlet to pass through the support platform;
k. a mechanical device connected to the platform for shaking, rotating, rocking or vibrating the platform;
l. a plurality of sensors in communication with an electronic controller to allow control of the condition of a liquid in the container.
m. a capture column disposed under the support platform and above ground, holding a binding resin and connected to the liquid outlet to receive liquid from the container further comprising a process liquid inlet, a process liquid outlet and a process liquid outlet control valve.

2. The bioreactor of claim 1, wherein the exhaust gas is allowed to pass through a Bunsen burner further comprising a source of a fuel gas and a source of oxygen to incinerate and decontaminate the exhaust gas, prior to venting the exhaust gas out to the environment.

3. The bioreactor of claim 1, wherein the container is round, square, rectangular, or oval in shape.

4. The bioreactor of claim 1, wherein the container is flexible.

5. The bioreactor of claim 1, wherein the container is comprised of plastic or metal.

6. The bioreactor of claim 1, wherein the inner volume of the container ranges from 10 mL to 2000 L.

7. The bioreactor of claim 1, wherein the container is maintained at a positive pressure differential of 0.03 to 0.05 inches water gauge with respect to environment by adjusting the speed of the vent fan and the one-way exhaust gas flow control valve closes when the pressure differential pressure between the container and the environment reaches below 0.03 inches water gauge.

8. The bioreactor of claim 1, wherein the inert gas is nitrogen or a noble gas.

9. The bioreactor of claim 1, wherein the temperature of the nutritive or inert gas is within 2-4 degrees of a pre-determined level by the inline heater or cooler disposed in the gas inlet.

10. The bioreactor of claim 1, wherein the capture column further comprises a plurality of flexible porous pouches having pores sizes ranging in size between 5 and 50 microns and capable of holding the binding and disposed on a plurality of porous hard surfaces separated from each other by gaskets disposed between the porous hard surfaces.

11. The bioreactor of claim 1, wherein the sparging element comprises at least one perforated flexible or inflexible unit wherein the size of perforations ranges from 1-100 microns, varying based on proximity to the gas inlet, wherein the pore sizes are smaller.

12. The bioreactor of claim 1, wherein the height of culture medium in the container ranges from 2 to 10 inches.

13. The bioreactor of claim 1 wherein the container is filled with culture medium and cell culture to occupy 30-70% of the inner volume of the container.

14. The bioreactor of claim 1, wherein the contents of in the container are passed through the capture column at the end of a bioprocess cycle to operate the bioreactor in a batch production mode; wherein the content in the container are continuously or periodically supplemented with nutritive elements, prior to allowing the content of the container to pass through the capture column to operate the bioreactor in a fed-batch production mode; wherein the liquid outlet further comprises a filter to hold the cell culture and allow the culture medium to pass through the capture column, and the removed culture medium is replaced periodically, intermittently or continuously with fresh culture medium to allow the bioreactor to operate in a perfusion production mode; wherein a pre-determined fraction of the cell culture and culture medium is removed from the container continuously, periodically or intermittently and replaced with an equivalent amount of fresh cell culture and fresh culture medium to operate the bioreactor in a continuous production mode; wherein a pre-determined average age of the cell culture is maintained at steady-state in the container by removing a pre-determined percentage of the cell culture from the container calculated by an equation 100*(1/pre-determined average age).

15. A method for expressing and capturing a biological product comprising:
a. providing a bioreactor according to claims 1;
b. introducing into the container an appropriate volume of culture medium;
c. starting flow of a nutritive gas pre-heated to a temperature equal to or 2-4 degrees higher than a pre-determined temperature of the culture medium;
d. introducing a pre-determined amount of cell culture in the container after the culture medium reaches a predetermined temperature;
e. adjusting pH and concentration of nutritive gas or gasses continuously, periodically, or intermittently to pre-determined levels;
f. continue operation of bioreactor for a pre-determined time to express a pre-determined quantity of a biological product;
g. providing a binding resin specific to the expressed product in the capture column;
h. opening the liquid outlet and allowing the content of the container to flow through the capture column;
i. monitoring concentration of biological product in the liquid flowing out of the capture column and closing the liquid outlet when the concentration of the biological product in the liquid flowing out reaches a pre-determined level;
j. introducing a washing liquid through the process liquid inlet in the capture column and allowing it to pass through the capture column until the liquid flowing out of the capture column meets a pre-determined level of debris and cell culture;
k. introducing an eluting liquid through the process liquid inlet in the capture column and allowing it to pass through the capture column until the liquid flowing out of the capture column meets a pre-determined level of biological product;
l. closing the process liquid inlet;
m. opening the liquid outlet and repeating the steps (i) through (l); and
n. collecting and cumulating the liquid flowing out in step (k) for further purification.

16. The method of claim 15, wherein in step (e), a pre-determined level of a solution of nutrients is introduced continuously, periodically or intermittently to operate the bioreactor in a fed-batch production mode.

17. The method of claim 15, wherein step (h) is started simultaneously to the operation of the bioreactor allowing continuous flow of the cell culture and culture medium from the container and replacing with an equivalent quantity of fresh cell culture and fresh culture medium to operate the bioreactor in a continuous production mode.

18. A method for optimizing bioprocess conditions for expression and capture of a biological product:
a. providing a bioreactor according to claim 1;
b. establishing a DOE plan and determining a pre-determined number of experiments required for optimizing conditions of bioprocessing;
c. disposing a plurality of containers on the support platform a determined in step (b);
d. adjusting condition of the content of each container as pre-determined in step (b);
e. operating bioreactor;
f. collecting expressed biological product;
g. evaluating optimal conditions to yield optimal quality and quantity of biological product.

19. A method for scaling up and producing a biological product expressed by a cell culture comprising expressing a biological product in a plurality of containers of an inner volume wherein an optimal quality and titer of a biological product is produced and combining the yield from each of the plurality of the containers to create a batch.
